(19) United States
(12) Patent Application Publication
(10) Pub. No.: US 2008/0120756 A1
(43) Pub. Date: May 29, 2008

(54) Protective Article

(75) Inventor: Steven H. Shepherd, West Palm Beach, FL (US)

Correspondence Address: AKERMAN SENTERFITT, P.O. BOX 3188, WEST PALM BEACH, FL, 33402-3188, US

(73) Assignee: Ringstar, Inc., West Palm Beach, FL (US)

(21) Appl. No.: 11/563,833

(22) Filed: Nov. 28, 2006

(51) Int. Cl. A41D 13/00; A41D 27/26; A61F 5/00; A61L 15/00; A61F 13/00

(52) U.S. Cl. 2/22; 2/267; 602/23; 602/75; 602/60

(57) ABSTRACT

A protective article comprising: a leg protective segment extending at least over a portion of the shin of the wearer; a midfoot protective segment extending over at least a portion of the dorsal side of the wearer's midfoot, wherein the midfoot protective segment is attached to the leg protective segment; a toe protective segment extending over at least a portion of the wearer's toes, wherein the toe protective segment is attached to the midfoot protective segment; and at least one securing structure, wherein the at least one securing structure securing the protective segments to the leg, foot, and toes of the wearer.

### CROSS REFERENCE TO RELATED APPLICATIONS

(Not Applicable)

### STATEMENT REGARDING FEDERALLY SPONSORED RESEARCH OR DEVELOPMENT

(Not Applicable)

### BACKGROUND OF THE INVENTION

1. Field of the Invention

The present invention relates to a wearable protective article, and more particularly to a protective article that can be used during physical activities such as contact sports.

2. Description of the Related Art

The lower extremities can often be subjected to impacts during various physical activities. Existing leg protective devices in the art are primarily constructed to provide protection for the shins and knees. For example, the toes, feet, and legs of construction workers are subject to impacts from heavy construction materials or tools. Similarly, delivery workers are also prone to similar impacts from heavy packages. Athletes also face similar problems. However, some sports and fitness activities increase the possibility of the participant to receive an impact on the feet or toes, such as martial arts, soccer, hockey, or field hockey. The toes, feet, and legs can sustain severe blows during such activities, especially in activities involving kicking maneuvers. In particular, because the natural tendency is to use at least a portion of the dorsal side of a foot and the front side of the leg to engage in kicking maneuvers, whether intentional or inadvertent, the dorsal side of the foot and the shin are more prone to receiving impacts. Additionally, injury can result from kicks landed against another person during certain activities, either inadvertently or, in the case of the martial arts, while sparring. For example, there is a possibility that the impact of the harder parts of the lower limbs, such as dorsal side of the midfoot or the shin portion of the lower leg, can injure the participants during the activity. It is therefore desirable to provide a leg protective device which protects both the wearer and the sparring partner or opponent from such injuries during activities such as martial arts and kickboxing. It is particularly desirable to provide such a protective device for use by children who may be learning martial arts.

Prior art leg protective devices adapted to prevent injury from kicking to both the wearer and an opponent are generally those designed specifically for use in the martial arts. Such devices generally consist of a shin guard specifically designed to cover only the lower leg and not the foot of the wearer. Other devices comprise of an instep guard specifically designed to cover only the midfoot of the wearer. Some devices combine both aspects, but provide no protection for the toes of the wearer. Devices designed to protect the toes of a wearer generally comprise a boot-type structure surrounding the entire foot and toes. However, such devices do not provide protection to the upper leg and extending the boot-type structure to protect the leg would be cumbersome and reduce the flexibility of the wearer.

It is generally known in the art that in order to provide lower limb protection and good flexibility, jointed protective devices adjacent to the sections of the lower limbs are preferred. For example, in baseball, a catcher's shin guard is constructed from jointed sections that protect the lower limbs of the wearer, yet still provide the wearer with maximum flexibility for the knees, ankles, and feet. However, such devices are not generally secured to the body of the wearer except at one or two support locations, since generally the catcher remains motionless during pitching of baseballs, and are designed to be removed easily and quickly. In such devices, the catcher's shin guard basically just rests on the body with minimal fastening to the catcher's body. As a result, such devices are not adapted to provide protection during continuous movement of the wearer or nor are they adapted to resist continuous and intentional impacts.

Therefore, what is needed is a protective article which can protect the legs, feet, and toes of a wearer in motion from impacts. More particularly, a protective article is needed that can be adapted for kicking activities, which can protect a wearer of the article, and in the case of marital arts, and also protect other participants from injury. However, there is also a need for such an article to still permit the wearer as much flexibility as possible in the movement of the feet, toes, and legs.

### SUMMARY OF THE INVENTION

The present invention is directed to a protective article that provides protection for the legs, feet, and toes of the wearer. The protective article can be flexible and easy to put on. The protective article can be used by itself or in concert with other foot coverings such as socks, stockings, and shoes.

In a first arrangement of the invention, a protective article is provided comprising a leg protective segment extending at least over a portion of the shin of the wearer; a midfoot protective segment extending over at least a portion of the dorsal side of the wearer's midfoot, wherein the midfoot protective segment is attached to the leg protective segment; a toe protective segment extending over at least a portion of the wearer's toes, wherein the toe protective segment is attached to the midfoot protective segment; and at least one securing structure, wherein the at least one securing structure secures the protective segments to the leg, foot, and toes of the wearer.

In some arrangements, the securing structure may comprise at least one leg segment securing structure, at least one midfoot segment securing structure, and at least one toe segment securing structure. In the various arrangements, at least one of the securing structures may comprise an elastic material.

In some arrangements a leg segment securing structure further comprises a leg wrap having at least one fastener and is operable to secure the leg protective segment adjacent to the wearer's shin. In some arrangements, a first end of the leg wrap is attached to the leg protective segment and a second end of the leg wrap has at least one fastener.

In some arrangements, a midfoot segment securing structure further comprises an arch strap extending from the midfoot protective segment and is operable to secure the midfoot protective segment adjacent to the dorsal side of the wearer's midfoot. In some arrangements, the arch strap is attached to the midfoot protective segment at a first end and a second end, where the arch strap is widthwise across the plantar side of the midfoot protective segment.

In some arrangements, a toe segment securing structure further comprises a toe strap extending from the toe protective segment and is operable to secure the toe protective segment adjacent to the dorsal side of the wearer's toes. In some arrangements, the toe strap is attached to the toe protective segment at a first end and a second end, where the toe strap is disposed widthwise on the plantar side of the toe protective segment. In other arrangements, a toe segment securing structure further comprises at least one toe loop extending from the toe protective segment and operable to secure the toe protective segment adjacent to the dorsal side of the wearer's toes.

In various arrangements, one or more of the segments may have padding material disposed therein. In some arrangements, the thickness of padding material may vary over the protective article. In some arrangements, the padding material may be contoured to generally match the contour of a wearer's feet and legs.

In some arrangements, the segments of the protective article are detachable from each other, allowing damaged portions to be replaced. Additionally, in other arrangements, the leg protective segment and/or the midfoot protective maybe further comprised of a plurality of detachable protective sections, where a total length of the leg protective segment or midfoot protective segment may be adjusted by removing one or more of the plurality of detachable protective sections.

In other arrangements, a toe bridge may be used to couple the toe protective segment and the midfoot protective segment together. In some arrangements, the toe bridge may comprise an elastic material for added flexibility.

### DESCRIPTION OF THE DRAWINGS

There are shown in the drawings arrangements which are presently discussed, it being understood, however, that the invention is not limited to the precise arrangements and instrumentalities shown, wherein:

FIG. 1 is a front view of a device in accordance with an arrangement of the invention.

FIG. 2 is rear view of a device in accordance with an arrangement of the invention.

FIG. 3 is a side view of a device in accordance with an arrangement of the invention.

FIG. 4 is a cross-section of a leg protective segment arrangement in accordance with an arrangement of the invention through the line 4-4 in FIG. 1

FIG. 5 is a cross-section of a midfoot protective segment arrangement in accordance with an arrangement of the invention through the line 5-5 in FIG. 1.

FIG. 6 is a cross-section of a toe protective segment arrangement in accordance with an arrangement of the invention through the line 6-6 in FIG. 1.

### DETAILED DESCRIPTION OF THE DRAWINGS

With reference now to the various figures in which like elements are identically numbered throughout, a description of the various arrangements of the present invention will now be provided. While the invention is disclosed in the context of a single arrangement, it will be appreciated that the invention can include numerous modifications from the preferred arrangement.

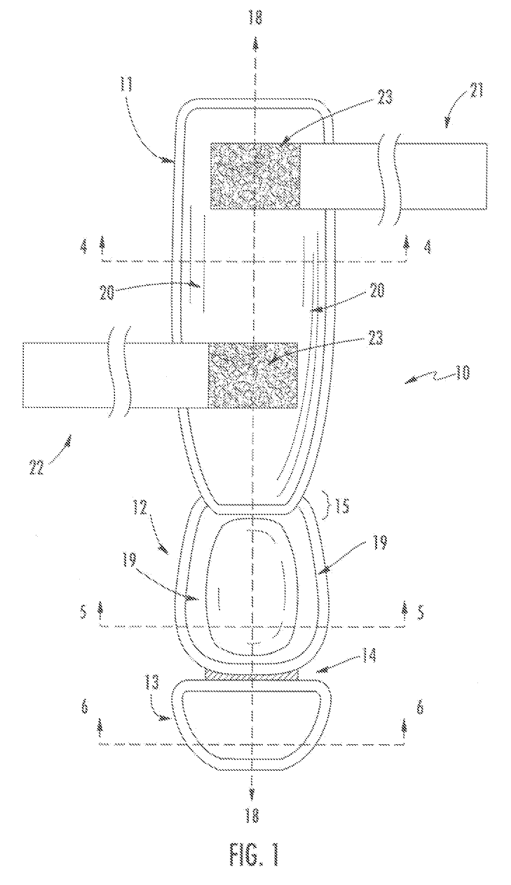

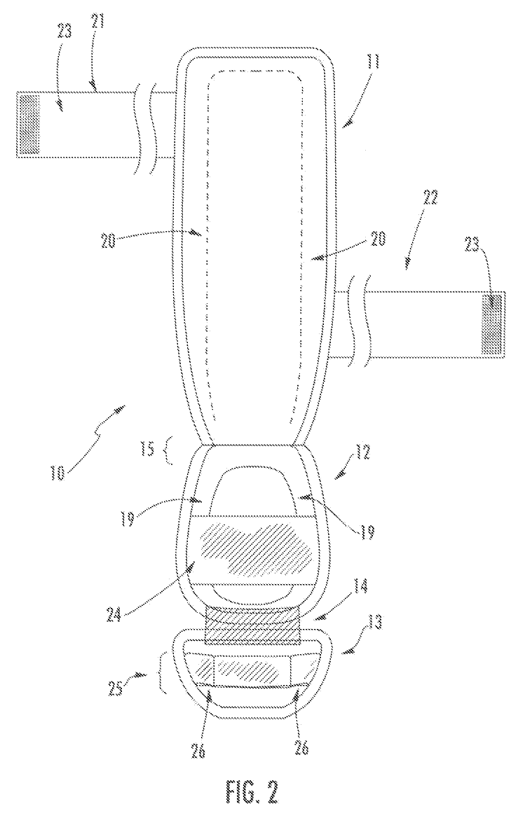

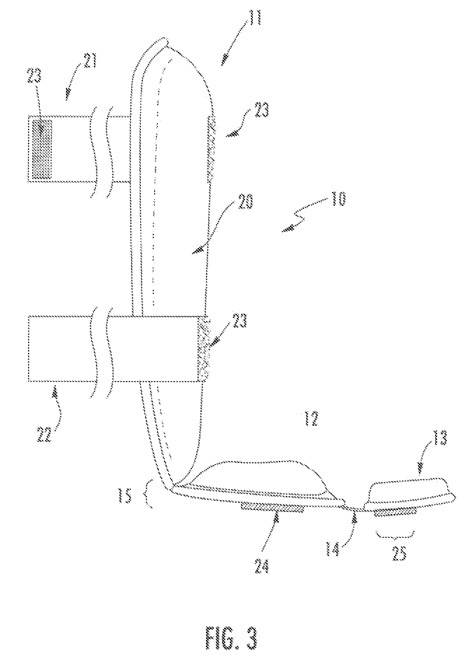

With reference to FIGS. 1-3, a protective article is shown at reference numeral 10. It should be understood that the protective article 10 can be used for a variety of activities in which protection of the toes, feet, and legs is necessary. In particular, the article 10 can be used for activities when it is desired to have fixed protection on the toes, feet, and legs. Such activities can include construction work, delivery work, or athletic activities that require continuous movement on the part of the participant, protection for the toes, feet, and legs, and optimal flexibility. For example, athletic activities, including martial arts sports such as karate, kickboxing, tae kwon do, and kung-fu, require maximum flexibility but can place great stress on the toes, feet, and legs during sparring or even training. One application of the protective article 10 is as a shin, foot, and toe guard for martial arts sports.

While the invention is described with respect to its use in martial arts, it should be appreciated that the device has application in any field where lower limb protection is required. The features of the illustrated arrangement of the protective article device 10 are particularly well adapted for martial arts, as protective article 10 provides protection from impacts for the toes, feet, and legs during training with bare feet. However, article 10 is suited for other activities, including non-martial arts activities, where impact protection for the toes, feet, and legs is sought. In such embodiments the protective article could be adapted for use with or without bare feet, socks, shoes, or any other type of foot coverings.

The protective article 10 comprises a plurality of segments constructed from one or more pliable materials such as leather, vinyl, or cloth. The material used for the segments can be sufficiently resistant to impact so that it can be used for sparring or punching bag training. In addition, because the skin will encounter moisture, primarily from sweat, the material used for the segments can comprise a material that can resist damage caused by contact with moisture. In some arrangements, a vinyl backed cloth can be used from some or all of the segments of the protective article 10. Preferably, the material includes an elastometric component, so that the material is at least partially stretchable. For Example, the material may include a spandex or elastane blend. Additionally, in other arrangements, one or more of the segments of the protective article 10 may be reinforced to protect stress areas from tearing apart or otherwise being damaged after repeated use or stress.

As previously stated, the protective article 10 comprises a plurality of segments. In the illustrated arrangement, as shown in FIGS. 1-3, the protective article 10 is comprised of at least three (3) segments. The first segment can be referred to as the leg protective segment 11, which provides protection to the lower leg of wearer. In the various arrangements, the leg protective segment 11 is adapted to extend over at least a portion of the shin of the wearer. The second segment can be referred to as the midfoot protective segment 12, which provides protection to the dorsal side of the midfoot of the wearer. In the various arrangements, the midfoot protective segment 12 is adapted to extend over at least a portion of the dorsal side of the midfoot of the wearer. The third segment can be referred to as the toe protective segment 13, which provides protection to the toes of the wearer. In the various arrangements, the toe protective segment 13 is adapted to extend over at least a portion of the dorsal side of the toes of the wearer.

In various arrangements, the leg protective segment 11 may be adapted to cover all or a portion of the lower leg of the wearer. In the illustrated arrangement in FIGS. 1-3, the leg protective segment 11 is configured to only cover the front portion of the leg, including the shin. In such arrangements, the leg protective segment 11 can be adapted to extend from a location below the knee to a location proximal to the ankle joint of the wearer. In such arrangements, complete coverage of the back of the lower leg may not be necessary where only the front side of the lower limb of the wearer is prone to injury.

In the various arrangements, the midfoot protective segment 12 may be adapted to cover all or a portion of the foot, excluding the toes. In the illustrated arrangement in FIGS. 1-3, the midfoot protective segment 12 is configured to only cover a portion of the dorsal side of the midfoot. In such arrangements, the midfoot protective segment can extend from a location proximal to the ankle joint of a wearer to a location proximal to the metatarsal-phalangeal joints of the wearer. In such arrangements, complete coverage of the foot may not be necessary where only the dorsal side of the midfoot of the wearer is prone to injury.

In the various arrangements, the toe protective segment may be adapted to cover all or a portion of the toes of the wearer. In the illustrated arrangement in FIGS. 1-3, the toe protective segment is configured to only cover the dorsal side of the toes of the wearer. In such arrangements, the toe protective segment can extend from a location proximal to the metatarsal-phalangeal joints of the wearer to the ends of the toes of the wearer. In such arrangements, complete coverage of the toes may not be necessary where only the dorsal sides of the toes of the wearer are prone to injury.

In the various arrangements, the segments of the protective device 10 may be joined in various ways. In the various arrangements, the segments of the protective device may be joined directly to each other, creating a joint, or by using a bridge to join the segments. In the illustrated arrangement in FIGS. 1-3, the midfoot protective segment 12 and the leg protective segment 11 are sewn directly together at to create a flexible midfoot joint 15. Furthermore, a first end and a second end of a toe bridge 14 are attached to the toe protective segment 13 and the midfoot protective segment 12, respectively, joining the two segments together. In the various arrangements, because flexibility is desired, a toe bridge 14 may be constructed from a pliable material, similar to those materials used for the segments of the protective article 10, to allow the joined segment to move easily with respect to each other. In some embodiments, a toe bridge may comprise elastic materials. In such arrangements, the elastic material allows the toe bridge 14 to flex more easily with respect to the midfoot protective segment 12 and to more naturally follow the contour of a wearer's foot and toes. Additionally, in arrangements where the segments are directly attached together, a seam, hinge point, or other type of flexing point, may be used to allow the joined segments of the protective article 10 to move easily with respect to each other.

It can be appreciated that in the various arrangements any combination of methods can be used to join the various segments of the protective article. In some arrangements, the segments of the protective article may be detachable from each other, allowing the wearer to replace damaged segments or remove segments that are not needed for a particular activity,

It can also be appreciated that the method used to attach particular segments together may be further adapted according to the demands of the particular joint. In these arrangements, a single bridge could be used to join all the segments together. For example, in some arrangements, the bridge could comprise a single strip of material, to which the segments are attached sequentially. In other arrangements, the bridge can comprise a sock-type structure with the protective segments attached thereto.

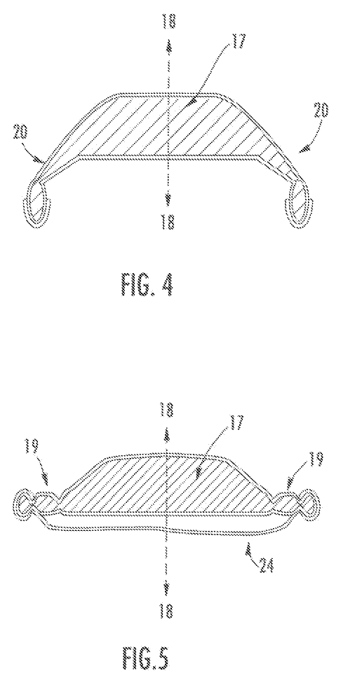

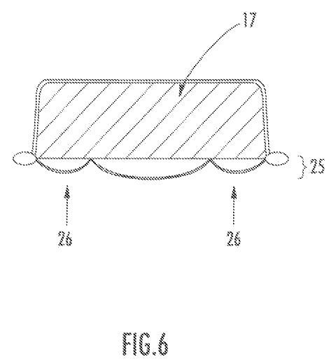

In the various arrangements, the segments of the protective device 10 may have padding material 17 disposed therein, as shown in the illustrated arrangements in FIGS. 4-6. For example, the padding material 17 can be any suitable material, for example, foam rubber, cotton, open-cell foam or closed-cell foam. The padding material 17 preferably has a high degree of resiliency and excellent shock absorption properties. In some arrangements, the padding material 17 is a closed-cell chemically cross-linked polyethylene or polyolefin foam, such as the Minicel® products manufactured by the Voltek Division of the Sekisui America Corporation. The closed-cell foams that can be used in the various arrangements and produced under the Minicel trademark include the L200, L300, L200F, L380, LS200, LS300, LS380, M200, M300, M380, MS200, MS300, MS380, T200, T300, TS200, TS300 and TS380 foams, which have excellent strength and shock absorption properties. In addition, these foams have a low degree of water absorption. Although not limited in this regard, closed-cell foam padding suitable for use in the segments of the protective article 10 preferably has a durometer of around 0.253. These foams also have a density of between approximately 1.5 and 4.5 pcf, a compression strength of between approximately 3 and 23 psi at approximately 25% deflection and between approximately 9 and 42 psi at approximately 50% deflection, and a tensile strength of between 28 and 145 psi. In some arrangements, the padding material may be arranged in layers, with foams of different types and thicknesses making up the different layers. For example, in one arrangement, a dense EVA foam may be layered between less dense closed cell foam layers.

In the various arrangements, the padding material 17 can be located throughout the segments of the protective article 10 or only in certain areas of the segments of the protective article 10. Furthermore, the padding material 17 can have an increased thickness or denser padding material 17 on those areas of the segments of the protective article 10 which cover portions of the foot that are more likely to be injured during athletic activities involving kicking, such as kickboxing and martial arts. For example, because there is a natural tendency for the top of dorsal side of the midfoot and the shin to be impacted, more padding or thicker padding may be placed on the top dorsal side of the midfoot protective segment 12 or along a central axis 18 of the leg protective segment 11. In the arrangement shown in FIGS. 1-5, such increased protection is provided by increasing the thickness of the padding material 18 in the leg protective segment and the midfoot protective segment 12 along an axis 18, as shown in FIGS. 4 and 5. In such arrangements, the padding material may be reduced elsewhere on the midfoot protective segment and the leg protective segment, as the risk of causing or receiving injury elsewhere is reduced.

In the various arrangements, the padding material 68 can have any suitable thickness, and may have a thickness of between 0.125 and 3 inches, such as 1 inch padding. In some arrangements, the padding material 68 may have a closed-cell foam padding core of 0.25 inch thickness covered by an open-cell foam padding of 1 inch thickness. The open cell foam padding is softer for increased comfort, but the closed-cell foam padding offers more injury protection. Areas of the protective article which are not thickly padded, such as the midfoot protective segment sides 19 or the leg protective segment sides 20, may be padded with any suitable thickness of padding, such as 1 inch, or may be replaced by an open mesh to allow for added ventilation of the foot of the wearer.

The padding material 17 disposed in the segments of the protective article 10 can comprise one general region of padding material 17 in each segment of the protective article 10, as shown in FIGS. 1-6. In some arrangements, the padding material 17 can comprise a single continuous piece of padding. The padding 18 can be of uniform or generally uniform thickness or can be of varying thickness, as previously described. In other arrangements, each segment of the protective article may have multiple pieces of padding material 17 disposed therein. In some arrangements the pieces of padding material 17 may be layered to provide additional thickness. The present disclosure also contemplates the use of two or more separate pieces of padding 18 that are connected or joined together in a single segment of the protective article 10. In such exemplary arrangements, the particular joining structures between the two or more separate pieces of padding material 17 can be chosen to provide adequate protection and comfort to the wearer. The present disclosure contemplates various sizes and shapes of the two or more pieces of padding material 17 being used in the segments of the protective article 10.

In other arrangements of the invention, it is contemplated that the padding material 17 is disposed on the surface of the segments of the protective article 10. In such arrangements, the padding material 17 can be protected from damage, by impact or by moisture, by using a protective casing or sheath. In some arrangements, the casing may be separate from the protective article 10 and attached later. In other arrangements, the casing may be defined by using multiple layers of material in the segments of the protective article 10 and defining a region in between some of the layers as the casing or sheath. In yet other arrangements, the casing may comprise an attached pad covering for the padding material 17 disposed on the segments of the protective article 10. In the various arrangements, the casing may comprise pliable materials, similar to the segments of the protective article 10, to match the segments of the protective article 10, or as necessary due to the location of the padding material 17. In some arrangements of the protective article 10, the casing covers all of the padding material 17; however the present disclosure contemplates the casing covering only a portion of the padding.

In the illustrated arrangement, it is contemplated that the segments of the protective article 10 comprise separate pieces joined together. However, this disclosure also contemplates that two or more of the segments may be formed from a single piece. For example, in the illustrated arrangement, the leg protective segment 11 and the midfoot protective segment 12 may be constructed from a single piece of material. Furthermore, the padding material 17 disposed therein may also be a single piece of material. In such arrangements, the midfoot joint 15 of the protective article 10 can be comprised of a flexible joint at the juncture between the midfoot protective segment and the leg protective segment 11. In some arrangements, a flexible midfoot joint 15 may be naturally formed when the padding material 17 comprises two separate padding regions, as discussed above. In other arrangements, a stitched seam through a single piece of padding material 17 may be used to define the flexible midfoot joint 15, which can act as a hinge without the need to provide two separate padding regions. The present disclosure contemplates other structures and techniques being used to provide a hinge along flexible joint 15 including decreasing the thickness of a continuous padding material 17 along the flexible joint region. The amount of the decreased thickness of the padding material 17 along the region of flexible joint 15 can be chosen based upon various factors including the amount of mobility that the flexible joint 15 is to allow.

In some arrangements, the shape of the segments of the protective article 10 may be adapted to conform to the general shape of the legs, feet, or toes. For example, in the illustrated arrangement shown in FIG. 4, the curvature of the leg protective segment is adapted to generally match the natural curvature of a wearer's leg. Furthermore, in the some arrangements, the midfoot protective segment 12 and the toe protective segment 13 may be adapted to cover a particular foot (left or right), while in other arrangements, as shown in the illustrated embodiment, a since protective article may be used on either foot.

In some arrangements, the segments of the protective article 10 and the padding material 17 disposed therein can be attached together by adhesive or sewn together. In other arrangements, the segments of the protective article 10 can constructed with a pocket or insert region in which the padding material 17 can be inserted. In other arrangements, the padding material 17 may be encased in the same material as the segments of the protective article 10.

In the various arrangements, the protective article may also comprise at least one securing structure adapted to secure each of the segments of the protective article 10 in place adjacent to the wearer's toes, feet, and legs, thus securing the protective article 10 in place. In the illustrated arrangement, each segment of the protective article 10 is held in place by a separate securing structure.

In some arrangements, a leg segment securing structure comprises at least one strap extending from a portion of the leg protective segment 11. In the illustrated arrangement, an upper leg strap 21 and lower leg strap 22 extend in opposite directions from locations on the front of the leg protective segment 11. The leg straps 21, 22 may also employ one or more fasteners 23 to fasten the leg protective segment 11 in place. The opposing forces of the oppositely placed leg straps assist the wearer in securing the leg protective segment 11, as the opposing forces prevent the secured end of the leg protective segment 11 from slipping while the second strap is secured in place.

In the various arrangements, the leg straps 21, 22 can be constructed from any suitable materials, including cloth, vinyl, or leather. The leg straps 21, 22 can also vary in width, according to their location. For example, in the illustrated embodiment, the upper strap 21 is 50% wider than the bottom strap 22. In such embodiments, the upper strap may be wider, as it lies adjacent to the naturally large and rounded calf muscle, and the increased surface area prevents slippage of the upper strap 21 and the leg protective segment 11. However, in such embodiments, the bottom strap 22 lies in a generally smaller and flatter region between the ankle and the calf muscle of the wearer, requiring less surface area to prevent slippage or the bottom strap 22.

In some arrangements, the leg straps 21, 22 can include elastic materials to better secure the leg protective segment 11 to the leg of the wearer. In the illustrated arrangement, a complementary set of fasteners 23 are disposed on a free end of the straps 21, 22 and on the attached end of the straps 21, 22. In the illustrated arrangement, the complementary fasteners 23 comprise hook and loop fasteners. In other arrangements, other types of fasteners may be used, however, hook and loop fasteners, by their construction, can reduce the risk of injury to the wearer and opponents due to a lack of sharp edges or hard materials. In the various arrangements, the leg segment securing structure is not limited to the illustrated arrangement. In some arrangements, the leg segment securing structure can comprise one or more long strips of material designed to wrap multiple times around the leg. In other arrangements, the leg protective segment and the leg segment securing structure may comprise a boot-type structure, elastic slip-on sock-type structure, or a clam-shell type structure.

In some arrangements, a midfoot segment securing structure comprises at least one strap extending from a portion of the midfoot protective segment 12. In the illustrated arrangement, an arch strap 24 extends widthwise from two opposites edges of the midfoot protective segment 12. In the various arrangements, the arch strap 24 can be constructed from any suitable materials, including cloth, vinyl, or leather. In some arrangements, the arch strap 24 can include elastic materials to better secure the midfoot protective segment 12 to the foot of the wearer and is most preferably an elastic strap stitched to the midfoot protective segment 12. However, it can be appreciated that in the various arrangements, the midfoot segment securing structure can comprise any of the same types securing structures previously described above for the leg segment securing structure.

In some arrangements, a toe segment securing structure comprises at least one strap extending from a portion of the toe protective segment 13. In the illustrated arrangement, a toe strap 25 extends widthwise across the plantar surface of the toe protective segment 13 from two opposite edges of the toe protective segment 13. In the various arrangements, the toe strap 25 can be constructed from any suitable materials, including cloth, vinyl, or leather. Preferably, the toe strap 25 can include elastic materials to better secure the toe protective segment 13 to the toes of the wearer and is most preferably an elastic strap stitched to the toe protective segment 13. In the illustrated arrangement, the toe strap is further adapted to comprise at least one toe loop 26. It can be appreciated that in the various arrangements including a toe loop 25, the toe loop 25 may be constructed in various ways. In the illustrated arrangement, the toe loop 26 is constructed by securing at least a portion of the length of the toe strap 25 to the plantar side of the toe protective segment 13. In such an arrangement, the first toe of a wearer may be inserted into one of the toe loops 26 and at least some of the other toes of the wearer may be inserted into the remaining portion of the toe strap 25 or the remaining toe loop 26, depending on the natural arrangement of the wearer's toes. The arrangement in the illustrated embodiment is advantageous, as the position of the toe loops 26, 26 allows the protective article 10 to be worn on either foot of the wearer and provide at least one toe loop 26 for the first toe on either foot of the wearer.

In other arrangements, at least one separate toe loop 26 may be attached to the plantar side of the toe protective segment 13. However, in addition to the securing structures described herein, it can be appreciated that in the various arrangements, the toe segment securing structure can comprise any of the same types securing structures previously described above for the leg segment securing structure.

It can be appreciated in the various arrangements, that the securing structure is not limited to those described above. In some arrangements, a single securing structure may be used for securing all the segments of the protective article 10 to the leg, foot, and toes of the wearer. For example, in some arrangements the securing structure could comprise a slip-on sock-type device, wherein each of the segments of the protective article are disposed on the sock device, wherein upon slipping the sock device onto the wearer's leg and foot, the segments of the protective article 10 are positioned over the appropriate regions of the foot. In other arrangements, at least one leg strap 22 can be adapted to have sufficient length to be wrapped around the leg, foot, and toes, thus securing the segments in place. These arrangements are presented by way of example and not by way of limitation.

In operation, as shown in the illustrated arrangement in FIGS. 1-6, the wearer inserts his foot into the protective article 10 at the midfoot protective segment 12. The arch strap 24 and the toe strap 25 initially secure the protective article 10 on the wearer's foot. Additionally, in arrangements with toe loops 26, the insertion of a wearer's toes into at least one of the toe loops 26 further secures the protective article 10 on the wearer's foot. Once the protective article 10 is secured on the foot and toes, the wearer can then position the leg protective segment 11 in place and first wrap the upper or the lower leg strap 21, 22 in place around the wearer's leg. In arrangements where elastic leg straps 21, 22 are used, the leg straps 21, 22 can be wrapped even tighter, allowing for a more secure fit. Similarly, a contoured leg protective segment, as show in FIGS. 1-4, also allows the leg straps 21, 22 to the wrapped more tightly, allowing for a more secure fit. Once the first leg strap is wrapped around the wearer's leg, the set of complementary fasteners 23 can hold the first leg strap in place. The wearer can then repeat the process for the remaining leg strap. Afterwards, the wearer can repeat the process on a second leg with a second protective article 10. Once the wearer no longer needs to use the protective article 10, the process can be reversed to remove the protective article 10.

It will be appreciated that the size of the protective article 10 can be altered to accommodate the different sizes associated with use by men, women, and children. The present disclosure also contemplates the use of various sizes and shapes for the segments of the protective article 10. For example, the length of the leg protective segment 11 can be increased or decreased, thus adapting the article for the needs of the particular wearer. The other segments and securing structures of the protective article 10 can similarly be adapted as well.

The present disclosure also contemplates that any of the segments of the protective article may be divided into a number of detachable sections of padding material 17 so that the protective article 10 can be customized for the particular needs of the wearer including particular sizes, particular activities and particular levels of protection by providing various interchangeable sizes and/or shapes and/or materials for the padding material 17 and the segments for the protective article 10.

While several arrangements of the invention have been described, it will be understood that it is capable of still further modifications and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

1. A protective article comprising:
a leg protective segment extending at least over a portion of the shin of the wearer;
at least one leg segment securing structure attached to the leg protective segment to secure the leg protective segment to the leg of a wearer;
a midfoot protective segment extending over at least a portion of the dorsal side of the wearer's midfoot, wherein the midfoot protective segment is coupled to the leg protective segment;
at least one midfoot segment securing structure attached to the midfoot protective segment to secure the midfoot protective segment to the foot of a wearer;
a toe protective segment extending over at least a portion of the wearer's toes, wherein the toe protective segment is coupled to the midfoot protective segment; and
at least one toe segment securing structure attached to the toe protective segment to secure the toe protective,
wherein the at least one toe segment securing structure comprises an elastometric toe strap extending from the toe padding, wherein the toe strap comprises least one toe loop separated from the remaining portion of the toe strap,
whereby the first toe of a wearer's foot may be inserted into the toe loop and at least some of the other toes may be inserted into the remaining portion of the toe strap.

2. (canceled)

3. The protective article of claim 1, wherein the at least one leg segment securing structure further comprises a leg wrap having at least one fastener and operable to secure the leg protective segment adjacent to the wearer's shin.

4. The protective article of claim 3, wherein a first end of the leg wrap is attached to the leg protective segment and a second end of the leg wrap has at least one fastener.

5. The protective article of claim 1, wherein at least one of the securing structures further comprises an elastic material.

6. The protective article of claim 1, wherein the at least one midfoot segment securing structure further comprises an arch strap extending from the midfoot protective segment and operable to secure the midfoot protective segment adjacent to the dorsal side of the wearer's midfoot.

7. The protective article of claim 6, wherein the arch strap is attached to the midfoot protective segment at a first end and a second end, the arch strap being further disposed on the plantar side of the midfoot protective segment.

8. (canceled)

9. The protective article of claim 1, wherein the toe strap is attached to the toe protective segment at a first end and a second end, the toe strap being further disposed on the plantar side of the toe protective segment.

10. The protective article of claim 1, wherein the toe strap is operable to secure the toe protective segment adjacent to the dorsal side of the wearer's toes.

11. The protective article of claim 1, wherein at least one of the protective segments has padding disposed therein.

12. The protective article of claim 11, wherein padding disposed in the leg protective segment is shaped to generally conform to the contour of the front of the wearer's leg.

13. The protective article of claim 11, wherein padding disposed in a first area of the leg protective segment is thicker than padding disposed in a second area of the leg protective segment.

14. The protective article of claim 11, wherein padding disposed in a first area of the midfoot protective segment is thicker than padding disposed in a second area of the midfoot protective segment.

15. The protective ankle of claim 1, wherein the midfoot protective segment is detachable from the leg protective segment.

16. The protective article of claim 1, wherein the toe protective segment is detachable from the midfoot protective segment.

17. The protective article of claim 1, wherein the leg protective segment is further comprised of a plurality of detachable protective sections, whereby a total length of the leg protective segment may be adjusted by removing one or more of the plurality of detachable protective sections.

18. The protective article of claim 1, wherein the midfoot protective segment is further comprised of a plurality of detachable protective sections, whereby a total length of the midfoot protective segment may be adjusted by removing one or more of the plurality of detachable protective sections.

19. The protective article of claim 1, further comprising a toe bridge coupling the toe protective segment to the midfoot protective segment.

20. The protective article of claim 19, wherein the toe bridge further comprises an elastic material.

21. The protective article of claim 1, wherein the toe strap is operable to secure the toe protective segment adjacent to the dorsal side of the wearer's toes, wherein the toe strap comprises two equally sized toe loops positioned on opposite ends of the toe strap, and a central section, wherein:
the first toe of either of a wearer's feet may be inserted into one of the toe loops;
at least two of the other toes of the wearer maybe inserted into the central section of the toe strap; and
the remaining other toes may be inserted into the second toe loop.
